(19) United States
(12) Patent Application Publication
(10) Pub. No.: US 2020/0289430 A1
(43) Pub. Date: Sep. 17, 2020

(54) Antibacterial Treatments Using Cannabinoids

(71) Applicant: Botanix Pharmaceuticals Limited, Northbridge Western Australlia (AU)

(72) Inventors: Matthew Callahan, Philadelphia, PA (US); Michael Thurn, New South Wales (AU)

(21) Appl. No.: 16/769,371

(22) PCT Filed: Nov. 16, 2018
(86) PCT No.: PCT/AU2018/051233

§ 371 Date: Jun. 3, 2020

Related U.S. Application Data
(60) Provisional application No. 62/594,380, filed on Dec. 4, 2017.

(30) Foreign Application Priority Data
Dec. 4, 2017 (AU) 2017904871

(51) Int. Cl. A61K 31/05

(52) U.S. Cl. CPC A61K 31/05; A61K 9/0014

(57) ABSTRACT

A method for the treatment or prevention of a topical infection by a bacteria of the genus Propionibacterium comprising the step of:

administering a therapeutically effective amount of a topical composition comprising a cannabinoid.

### TECHNICAL FIELD

A composition for the treatment or prevention of bacterial infections, comprising a cannabinoid, and a method for use thereof.

### BACKGROUND ART

Compounds with antimicrobial properties have attracted great interest in recent times as a result of an increase in the prevalence of infections caused by bacteria, resulting in serious or fatal diseases. Furthermore, the regular use of broad spectrum antibiotic formulas has led to the increased occurrence of bacterial strains resistant to some antimicrobial compositions.

Novel antimicrobial compounds have the potential to be highly effective against these types of treatment-resistant bacteria. The pathogens, having not previously been exposed to the antimicrobial composition, may have little to no resistance to the treatment.

There is no indication that bacterial resistance to antibiotics will stop and for this reason new antibiotics and new treatment options are necessary to achieve a desirable treatment outcome in patients.

Propionibacterium is a Gram-positive, anaerobic genus of bacteria named for their ability to synthesize propionic acid using transcarboxylase enzymes. Its members are primarily facultative parasites and commensals of humans and other animals, living in and around the sweat glands, sebaceous glands, and other areas of the skin. They are virtually ubiquitous and do not cause problems for most people, but propionibacteria have been implicated in acne and other skin conditions.

The present invention seeks to provide a new option for the treatment of bacterial infections, particularly skin conditions such as acne associated with Propionibacterium populations.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

According to one aspect of the invention, there is provided a topical composition comprising a cannabinoid for the treatment or prevention of an infection by a bacterium of the genus Propionibacterium. Preferably the topical composition is a pharmaceutical composition.

According to another aspect of the invention, there is provided a method for the treatment or prevention of a infection by a bacterium of the genus Propionibacterium in a subject in need of such treatment comprising the step of:
administering an effective amount of a topical composition comprising a cannabinoid.

According to another aspect of the invention, there is provided the use of a cannabinoid, in the manufacture of topical medicament for the treatment of an infection by a bacterium of the genus Propionibacterium in a subject.

Preferably the cannabinoid is cannabidiol. Preferably the bacterium is Propionibacterium acne. Preferably the infection is associated with acne, that is, bacterial growth by Propionibacterium is associated with bacterial carriage or a bacterial infection of the skin in the form of acne lesions.

### DESCRIPTION OF INVENTION

### Detailed Description of the Invention

According to one aspect of the invention, there is provided a topical composition comprising a cannabinoid for the treatment or prevention of an infection by a bacterium of the genus Propionibacterium. Preferably the topical composition is a pharmaceutical composition.

According to another aspect of the invention, there is provided a method for the treatment or prevention of an infection by a bacterium of the genus Propionibacterium in a subject in need of such treatment comprising the step of:
administering an effective amount of a topical composition comprising a cannabinoid.

According to another aspect of the invention, there is provided the use of a cannabinoid, in the manufacture of topical medicament for the treatment of an infection by a bacterium of the genus Propionibacterium in a subject.

Without being held to any theory, we believe that it is the resorcinol moiety of cannabinoids that serves as the antibacterial pharmacophore, with the alkyl, terpenoid, and carboxylic appendices modulating its activity.

The topical administration may comprise the administration of the therapeutically effective amount of a cannabinoid directly to a dermal or mucosal surface of the subject. Preferably, the cannabinoid is applied topically to the skin, mucosal membranes (oral, vaginal, rectal) or eye of the subject. The use may comprise administering a therapeutically effective amount of a cannabinoid, to the skin, mucosal membranes (oral, vaginal, rectal) or eye of a subject.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

Preferably, the composition of the present invention contains a cannabinoid at a concentration of: between 15 μg/mL and 0.1 μg/mL, 10 μg/mL and 1 μg/mL, 8 μg/mL and 2 μg/mL, or 3 μg/mL and 6 μg/mL.

Preferably, the composition of the present invention contains a cannabinoid at a concentration of: 0.1 μg/mL, 0.5 μg/mL, 1.0 μg/mL, 1.5 μg/mL, 2.0 μg/mL, 2.5 μg/mL, 3.0 μg/mL, 3.5 μg/mL, 4.0 μg/mL, 4.5 μg/mL, 5.0 μg/mL, 5.5 μg/mL, 6.0 μg/mL, 6.5 μg/mL, 7.0 μg/mL, 7.5 μg/mL, 8.0 μg/mL, 8.5 μg/mL, 9.0 μg/mL, 9.5 μg/mL, 10.0 μg/mL, 10.5 μg/mL, 11.0 μg/mL, 11.5 μg/mL, 12.0 μg/mL, 12.5 μg/mL, 13.0 μg/mL, 13.5 μg/mL, 14.0 μg/mL, 14.5 μg/mL, or 15.0 μg/mL.

Preferably the composition of the present invention delivers a therapeutically effective amount of the cannabinoid to the dermal or mucosal surface of the subject.

The phrase “therapeutically effective amount” as used herein refers to an amount of the cannabinoid sufficient to inhibit bacterial growth associated with bacterial carriage or a bacterial infection of the skin. That is, reference to the administration of the therapeutically effective amount of a cannabinoid according to the methods or compositions of the invention refers to a therapeutic effect in which substantial bacteriocidal or bacteriostatic activity causes a substantial inhibition of the bacterial carriage or bacterial infection of the skin. The term “therapeutically effective amount” as used herein, refers to a nontoxic but sufficient amount of the composition to provide the desired biological, therapeutic, and/or prophylactic result. The desired results include elimination of bacterial carriage or reduction and/or alleviation of the signs, symptoms, or causes of a disease, or any other desired alteration of a biological system. An effective amount in any individual case may be determined by one of ordinary skill in the art using routine experimentation. In relation to a pharmaceutical composition, effective amounts can be dosages that are recommended in the modulation of a diseased state or signs or symptoms thereof. Effective amounts differ depending on the pharmaceutical composition used and the route of administration employed. Effective amounts are routinely optimized taking into consideration various factors of a particular patient, such as age, weight, gender, etc and the area affected by disease or disease causing microorganisms.

Preferably, the bacterium is Propionibacterium acnes.

The subject may be any subject capable of colonisation by bacteria, such as bacteria of the genus Propionibacterium. The subject may be mammalian or avian. Preferably, the subject is selected from the group comprising human, canine, avian, porcine, bovine, ovine, equine, and feline. Most preferably, the subject is selected from the group comprising human, bovine, porcine, equine, feline and canine.

In one embodiment of the invention, the cannabinoid is administered to the subject using a dosing regimen selected from the group consisting of: three times daily; two times daily; daily; every second day, every third day, once weekly; once fortnightly and once monthly.

The pharmaceutical composition may optionally include a pharmaceutically acceptable excipient or carrier. Preferably, the composition is adapted to treat a topical bacterial infection.

The composition of the invention may be provided in a form selected from the group comprising, but not limited to, a rinse, a shampoo, a lotion, a gel, a leave-on preparation, a wash-off preparation, and an ointment. Preferably, the composition is selected from the group consisting of: an immediate release composition, a delayed release composition, a controlled release composition and a rapid release composition.

The composition of the invention may further comprise a second antimicrobial agent. The further antimicrobial agent may be an antifungal agent. For example, the composition may further comprise benzoyl peroxide, erythromycin, clindamycin, doxycycline or meclocycline.

The composition of the invention may further comprise an anti-inflammatory agent (such as a corticosteroid), an anticomedolyic agent (such as tretinoin), and/or a retinoid or derivative thereof.

The compositions described herein may be formulated for topical administration by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

Compositions of the invention may be administered topically. Therefore, contemplated for use herein are compositions adapted for the direct application to the skin. The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the invention may be provided in the form of a water-based composition or ointment which is based on organic solvents such as oils. Alternatively, the compositions of the invention may be applied by way of a liquid spray comprising film forming components and at least a solvent in which the cannabinoids are dispersed or solubilised. The administration of the cannabinoids in accordance with the methods and compositions of the invention may be by any suitable means that results in an amount sufficient to treat a microbial infection on a subject's skin or to reduce microbial growth at the location of infection. The cannabinoid may be contained in any appropriate amount and in any suitable carrier substance, and is generally present in an amount of 1-95% by weight of the total weight of the composition. The pharmaceutical or veterinary composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18th Edition, Mack Publishing Company, Easton, Pa., USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

Various topical delivery systems may be appropriate for administering the compositions of the present invention depending up on the preferred treatment regimen. Topical compositions may be produced by dissolving or combining the cannabinoids of the present invention in an aqueous or non-aqueous carrier. In general, any liquid, cream, or gel or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc, which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the cannabinoids are added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the cannabinoids can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the cannabinoids are added to the composition at the desired concentration at a point preceding addition of the gelling agent.

The amount of antibiotic compounds incorporated into a topical composition is not critical; the concentration should be within a range sufficient to permit ready application of the composition such that an effective amount of the cannabinoids is delivered.

The composition may be in the form of a controlled-release composition may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

Compositions of the invention may be in the form of a solid dispersion.

Preferably, the topical compositions of the present invention are used to treat or prevent acne. Unless the context requires otherwise, the term ‘acne’, as used herein, means one or more of: acne vulgaris, neonatal and infantile acne, perioral dermatitis, acne conglobata, hidradenitis suppurative, acne filminans, pyoderma faciale, acne excoriee des jeunes filles, acne mechanica, acne tropicalis, acne aestivalis, favre-racouchot syndrome, drug-induced acne, acne cosmetica, pomade acne, occupational acne, chloracne, steroid acne, rosacea, acne keloidalis nuchae and Gram-negative folliculitis.

### Kits

The invention also provides kits for use in the instant methods. Kits of the invention include one or more containers comprising a cannabinoid described herein, and instructions for use in accordance with any one of the methods described herein. The kit may further comprise a description of selecting an individual suitable for treatment based on identifying whether that individual has an infection by a bacterium of the genus Propionibacterium. The kit may further comprise a description of administering a cannabinoid described herein to an individual at risk of developing an infection by a bacterium of the genus Propionibacterium.

The instructions generally include information as to dosage, dosing schedule, and route of administration for the intended treatment. The containers may be unit doses, bulk packages (e.g. multiOdose packages) or sub-unit doses. Instructions supplied in the kits of the invention are typically written instructions on a label or package insert. The label or package insert indicates that the composition is used for treating, ameliorating and/or preventing an infection by a bacterium of the genus Propionibacterium. Instructions may be provided for practising any of the methods described herein.

### General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference, which means that it should be read and considered by the reader as part of this text. That the document, reference, patent application or patent cited in this text is not repeated in this text is merely for reasons of conciseness. No admission is made that any of the references constitute prior art or are part of the common general knowledge of those working in the field to which this invention relates.

Throughout this specification, unless the context requires otherwise, the term antimicrobial is understood to include compounds with antibacterial properties.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### EXAMPLES

### Example 1

This study, a Minimum Inhibitory Concentration (MIC), will be performed based upon the Macrodilution Broth Method outlined in CLSJ Document M07-A 10, Methods for Dilution Antimicrobial Susceptibility Tests for Bacteria that Gro111 Aerobically, Tenth Edition, and CLSI Document MI I-A8, Methods for Antimicrobial Susceptibility Testing of Anaerobic Bacteria, Eighth Edition.

The Test Material will be evaluated versus at total of 17 different microorganism strains: 7 strains of Methicillin resistant Staphylococcus aureus, 6 stains of Propionibacterium acnes, 2 strains of Erythromycin resistant Propionibacterium acnes, and 2 strains of Tetracycline resistant Propionibacterium acnes. Positive Control Material #1 will be evaluated versus 2 strains of Erythromycin resistant Propionibacterium acnes. Positive Control Material #2 will be evaluated versus 2 strains of Tetracycline resistant Propionibacterium acnes.

A suspension of each challenge strain will be prepared and exposed to each of 10 doubling dilutions of the test material or positive control materials prepared in the appropriate nutrient broth (reference Table I). Following incubation, the Minimum Inhibitory Concentration (MIC) of the test material and positive control materials will be determined visually and documented.

### Test Material and Control Material

Cannabidiol Lot
Erythromycin
Tetracycline HCl
   Staphylococcus aureus Preparation

2 to 4 days prior to testing, inocula from lyophilized vials or cryogenic stock cultures containing each species will be suspended in 0.9% Sodium Chloride Irrigation, USP (SCI), inoculated onto the surface of Tryptic Soy Agar (TSA) contained in Petri plates, and incubated at 35±2° C. for 24 to 48 hours, or until sufficient growth is observed.

24 to 48 hours prior to testing, growth from the agar plates previously prepared will be suspended in SCI.

The purity of each suspension will be verified by preparing isolation streaks of each culture on TSA. Aliquots of each suspension will then be spread-plated onto the surface of additional plates of TSA and incubated at 35±2° C. until sufficient growth is observed. This will produce lawns of the bacteria on the surface of the agar plates, and growth from these will be used to prepare the challenge suspensions.

Immediately prior to testing, an initial suspension of each species will be prepared in SCI by suspending the microorganisms from the solid media to achieve initial suspension concentrations of approximately 10⁸ CFU/mL. The suspensions may be centrifuged, if deemed necessary, to achieve the desired concentration.

Propionibacterium acnes Testing

2 to 6 days prior to testing, sterile tubes containing Reinforced Clostridial Medium (RCM) will be inoculated from lyophilized vials or cryogenic stock cultures containing these species. The broth cultures will be incubated anaerobically at 35±2° C. for 1 to 3 days, or until sufficient growth is observed.

Once sufficient growth is observed, the broth cultures will be subcultured into additional tubes of RCM (reference Table I) and incubated anaerobically at 35±2° C. The purity of each broth culture will be verified by preparing duplicate isolation streaks on Chocolate Agar with Enrichment (CAE), and incubating one plate aerobically and one anaerobically at 35±2° C.

Following incubation, initial suspensions containing approximately 10⁸ CFU/mL will be prepared for each species by centrifuging the broth culture tubes, combining the resulting pellets, and resuspending them in additional broth.

### Challenge Suspensions

Final Challenge Suspensions containing approximately 1×10⁶ CFU/mL of each microorganism strain will be prepared by transferring aliquots of the 10⁸ CFU/mL initial suspensions into separate containers containing a volume of the appropriate broth media sufficient to the needs of testing.

### Preparation of Test Material Dilutions

On each day of testing, the Test Material will be diluted (v/v) using the appropriate broth media to produce an intermediate solution containing 1000 μg/mL of Cannabidiol.

A series of 1:2 (v/v) dilutions of intermediate Test Material solution will be prepared using the appropriate broth media, to produce active concentrations of 100 μg/mL, 50 μg/mL, 25 μg/mL, 12.5 μg/mL, 6.25 μg/mL, 3.125 μg/mL, 1.562 μg/mL, 0.781 μg/mL, 0.390 μg/mL, and 0.195 μg/mL.

A 1.0 mL aliquot of the intermediate Test Material solution (100 μg/mL), and 1.0 mL aliquots of each of the 9 test material dilutions prepared will be transferred to separate sterile test tubes. For each challenge microorganism, three sets of test tubes will be prepared for the Test Material.

### Preparation of Positive Control Materials

A stock solution of each Positive Control Material containing 40960 μg/mL will be prepared in the appropriate solvent. The stock solutions will be dispensed into small containers and may be stored at −60° C., or greater, for up to six months. Individual containers of stock solution will be thawed immediately prior to testing, and will be discarded at the end of each test day.

On each day of testing, the Positive Control Material stock solutions will be diluted in the appropriate broth media to produce active concentrations of 4096 ug/ml, 2048 μg/mL, 1024 μg/mL, 512 μg/mL, 256 μg/mL, 128 μg/mL, 64 μg/mL, 32 μg/mL, 16 μg/mL, and 8 μg/mL.

A 1.0 mL aliquot of each Positive Control Material dilution prepared as described in Section 14.6 will be transferred to separate sterile test tubes. For each challenge microorganisms, three sets of test tubes will be prepared for each Positive Control material.

### Inoculation of Test Material and Positive Control Material Dilutions

A 1.0 mL aliquot of a challenge suspension containing approximately 1×10⁶ CFU/mL will be dispensed into each dilution tube in each series.

Following inoculation, final active Test Material concentrations of 50 μg/mL, 25 μg/mL, 12.5 μg/mL, 6.25 μg/mL, 3.125 ug/ml, 1.562 μg/mL, 0.781 μg/mL, 0.390 ug/ml, 0.195 μg/mL and 0.0976 μg/mL will be produced. Final active Positive Control Material concentrations of 2048 μg/mL, 1024 μg/mL, 512 ug/ml, 256 μg/mL, 128 μg/mL, 64 μg/mL, 32 ug/ml, 16 μg/mL, 8 ug/ml, and 4 ug/ml, will be produced.

Each dilution tube will contain approximately 5×10⁵ CFU/mL of the challenge microorganism.

### Controls

Positive Control Tubes (Growth Controls) containing a 1.0 mL aliquot of the appropriate broth media, and a 1.0 mL aliquot of challenge suspension will be prepared using each microorganism strain.

Negative Control Tubes (Sterility Controls; no microbial inoculation) of each broth media will be prepared.

### Incubation

The inoculated test material and positive control material dilution tubes and all controls will be incubated at 35±2° C. under the appropriate conditions for the times specified in Table 1, or until good growth is apparent in the positive control tubes.

### Determination of MIC Results

Following incubation, the tubes will be examined for growth of the challenge microorganism, as determined visually on the basis of turbidity.

The Minimum Inhibitory Concentration (MIC) of the test material or positive control materials versus each microorganism strain will be recorded as the highest dilution (lowest active concentration) that completely inhibits growth of the microorganism, as detected by the unaided eye.

TABLE 1
Challenge Microorganisms, media and incubation conditions

| | | | Incubation | | Incubation Temperature | |
|---|---|---|---|---|---|---|
| | Incubation | Incubation | Time Initial | Incubation | Inoculum Cultures and | |
| Microorganism | Time Inoculum | Time | Population | Temperature | Initial Population | Growth Media |
| Species | Cultures | MIC Tubes | Determination plates | MIC Tubes | Determination plates | (Inoculum¹/IP²/MIC³) |
| Propionibacterium | 48 to 72 | 46 to 48 | 48 to 72 | 35 ± 2° C. | 35 ± 2° C. | RCM¹/RCA²/RCM³ |
| acnes strains | hours | hours | hours | Anaerobic | Anaerobic | |
| Staphylococcus | 18 to 24 | 16 to 20 | 48 to 72 | 35 ± 2° C. | 35 ± 2° C. | TSA¹/TSA²/CAMHB³ |
| aureus strains | hours | hours | hours | | | |

Note:
Incubation times are nominal, but in practice, incubation will continue until good growth is observed.

### Results

The MIC for the test microorganisms were as follows.

TABLE 2
Minimum Inhibitory Concentration of Cannabidiol
against Propionibacterium acnes

| | | Minimum Inhibitory |
|---|---|---|
| | | Concentration (MIC) |
| Microorganism species | ATCC # | Cannabidiol (μg/mL) |
| Propionibacterium acnes | #6919 | 3.125 |
| Propionibacterium acnes | #6922 | 6.25 |
| Propionibacterium acnes | #6923 | 6.25 |
| Propionibacterium acnes | #11827 | 3.125 |
| Propionibacterium acnes | #11828 | 6.25 |
| Propionibacterium acnes | #51277 | 6.25 |

TABLE 3
Minimum Inhibitory Concentration of Cannabidiol
against Propionibacterium acnes Erythromycin
Resistant Clinical Isolates

| | | Minimum Inhibitory | |
|---|---|---|---|
| | | Concentration | |
| | | (MIC) (μg/mL) | |
| Microorganism species | BSLI# | Cannabidiol | Erythromycin |
| Propionibacterium acnes | #281014Pa5 | 6.25 | 2048 |
| Propionibacterium acnes | #281014Pa6 | 6.25 | 2048 |

TABLE 4
Minimum Inhibitory Concentration of Cannabidiol
against Propionibacterium acnes Tetracycline
Resistant Clinical Isolates

| | | Minimum Inhibitory | |
|---|---|---|---|
| | | Concentration | |
| | | (MIC) (μg/mL) | |
| | | | Tetracycline |
| Microorganism species | BSLI# | Cannabidiol | HCl |
| Propionibacterium acnes | #281014Pa2 | 3.125 | 64 |
| Propionibacterium acnes | #281014Pa4 | 6.25 | 128 |

TABLE 4
Minimum Inhibitory Concentration of Cannabidiol against
Methicillin resistant Staphylococcus aureus

| | | Minimum Inhibitory |
|---|---|---|
| | | Concentration (MIC) |
| Microorganism species | ATCC # | Cannabidiol (μg/mL) |
| Staphylococcus aureus | #BAA-41 | 1.562 |
| Staphylococcus aureus | #BAA-42 | 1.562 |
| Staphylococcus aureus | #BAA-44 | 1.562 |
| Staphylococcus aureus | #BAA-1683 | 1.562 |
| Staphylococcus aureus | #BAA-2094 | 1.562 |
| Staphylococcus aureus | #BAA-2313 | 1.562 |
| Staphylococcus aureus | #33592 | 1.562 |

Thus it can be seen that the MIC for cannabidiol against Propionibacterium acnes is between 3.125 μg/mL and 6.25 μg/mL, even in relation to strains that are resistance to other antibiotics. The MIC for cannabidiol against Staphylococcus aureus is about 1.562 μg/mL.

1. A method for the treatment of a topical infection by a bacteria of the genus Propionibacterium comprising administering a therapeutically effective amount of a topical composition comprising a cannabinoid at a concentration of between 3 μg/mL and 6 μg/mL.

2. A topical composition comprising a cannabinoid for the treatment or prevention of a topical infection by a bacterium of the genus Propionibacterium.

3. Use of a cannabinoid in the manufacture of topical medicament for the treatment of an infection by a bacterium of the genus Propionibacterium in a subject.

4. The method of claim 1 wherein the cannabinoid is chosen from the list comprising: cannabidiol, cannabinol, cannabigerol, cannabichromene, and Δ⁹-tetrahydrocannabinol.

5. The method of claim 1 wherein the cannabinoid is cannabidiol.

6. (canceled)

7. The method of claim 1 wherein the cannabinoid is delivered at a concentration of: 0.1 μg/mL, 0.5 μg/mL, 1.0 μg/mL, 1.5 μg/mL, 2.0 μg/mL, 2.5 μg/mL, 3.0 μg/mL, 3.5 μg/mL, 4.0 μg/mL, 4.5 μg/mL, 5.0 μg/mL, 5.5 μg/mL, 6.0 μg/mL.

8. The method of claim 1 wherein the bacteria is Propionibacterium acnes.

9. A kit comprising one or more containers comprising a topical composition comprising a cannabinoid, and instructions for use of the topical composition comprising a cannabinoid for the treatment or prevention of a topical infection by a bacteria of the genus Propionibacterium.
